(19) United States
(12) Patent Application Publication
(10) Pub. No.: US 2004/0033532 A1
(43) Pub. Date: Feb. 19, 2004

(54) Use of three-dimensional crystal structure coordinates to design and synthesize domain-selective inhibitors for angiotensin-converting enzyme (ACE)

(76) Inventors: Mario R. W. Ehlers, Mercer Island, WA (US); Barton Holmquist, Eagle, NE (US)

Correspondence Address: MARIO R. EHLERS, 7927 EAST MERCER WAY, MERCER ISLAND, WA, 98040, US

(21) Appl. No.: 10/637,430

(22) Filed: Aug. 8, 2003

Related U.S. Application Data
(60) Provisional application No. 60/401,971, filed on Aug. 8, 2002.

(51) Int. Cl. G01N 33/53; G06F 19/00; G01N 33/48; G01N 33/50

(52) U.S. Cl. 435/7.1; 702/19

(57) ABSTRACT

It has now been discovered that the use of the three-dimensional crystal structure coordinates of angiotensin-converting enzyme (ACE) will enable the design and synthesis, by means of computational chemistry and structure-guided drug design, of inhibitors of ACE that are highly selective and specific for either the N domain or the C domain of the enzyme, for the treatment of diverse diseases. The invention also relates to methods and processes for the structure-guided design and synthesis of dual N- and C-domain ACE inhibitors, and inhibitors that operate by competitive, non-competitive, uncompetitive, and irreversible mechanisms.

### CROSS REFERENCE TO A RELATED APPLICATION

This application claims priority to U.S. provisional application No. 60/401,971 filed Aug. 8, 2002.

### STATEMENT REGARDING FEDERALLY SPONSORED RESEARCH

Not Applicable.

### FIELD OF THE INVENTION

The present invention relates to the use of three-dimensional crystal structure coordinates to design, by means of computational chemistry and structure-guided drug design, N- and C-domain-selective angiotensin-converting enzyme (ACE) inhibitors for the treatment of diverse diseases.

### BACKGROUND OF THE INVENTION

Angiotensin-converting enzyme (ACE) is a key regulatory enzyme in the cardiovascular system. ACE is a critical component of the renin-angiotensin system (RAS), which controls blood pressure and strongly influences the function of the heart and the kidneys, as well as the walls of blood vessels. For these reasons, drugs that target the RAS—including ACE inhibitors, angiotensin II receptor blockers, and aldosterone antagonists—are among the most important therapeutic agents available today for the treatment of hypertension, heart failure, renal insufficiency, and general atherosclerosis [P. Sleight (2002) The renin-angiotensin system: a review of trials with angiotensin-converting enzyme inhibitors and angiotensin receptor blocking agents. Eur. Heart J. Suppl. 4: A53-A57; T. Unger (2002) The role of the renin-angiotensin system in the development of cardiovascular disease. Am. J. Cardiol. 89: 3A-9A].

Current-generation ACE inhibitors were developed in the late 1970s and early 1980s, and include captopril, enalapril, and lisinopril, among others. The design of these drugs was the result of guesswork, as neither the sequence nor the structure of ACE was known at the time.

Based on the serendipitous finding that several snake venom peptides were potent inhibitors of ACE and on a presumed similarity between ACE and the better-studied enzyme CPD-A, the early ACE inhibitors were born [D. W. Cushman & M. A. Ondetti (1999) Design of angiotensin converting enzyme inhibitors. Nat. Med. 5: 1110-1113]. Nevertheless, within a few years of their launch in the early 1980s, the current-generation ACE inhibitors rapidly established themselves as effective agents for the treatment of hypertension and heart failure and quickly became billion-dollar drugs.

Despite the success of the early ACE inhibitors, many patients (up to 20%) are unable to tolerate long-term treatment with current-generation ACE inhibitors because of side effects, most commonly a persistent dry cough. There are also occasional instances of a more serious adverse effect known as angioedema, which can be life-threatening [U. M. Steckelings et al. (2001) Angiotensin-converting enzyme inhibitors as inducers of adverse cutaneous reactions. Acta Derm. Venereol. 81: 321-325]. These adverse effects are likely related to the fact that treatment with current-generation ACE inhibitors not only inhibits the production of angiotensin II but also affects the levels of other active peptides, some not well understood. A further problem is that in some patients, chronic therapy with current-generation ACE inhibitors results in reduced efficacy over time, sometimes termed an “ACE inhibitor escape phenomenon,” indicating that the potency of current-generation ACE inhibitors can be improved by, for instance, the design of irreversible ACE inhibitors.

All current-generation ACE inhibitors are reversible, competitive-type, active site-directed inhibitors. Moreover, ACE was recently shown to consist of two similar but non-identical domains (N and C domains) that each contain an active site with different activities.

To improve on the therapeutic efficacy and side effect profile of current-generation ACE inhibitors, requires high-resolution structural data for the enzyme. This will enable rational design of domain-selective inhibitors, which will comprise the next-generation ACE inhibitors.

Despite intensive efforts by numerous academic and industry research groups over many years, the ACE crystal structure could not be solved. This has largely been due to the inability to generate ACE proteins, from natural or recombinant sources, that can yield crystals suitable for high-resolution x-ray diffraction. It is anticipated that once ACE proteins can be crystallized, the three-dimensional x-ray structure can be determined and solved rapidly. Recent developments indicate that proteins suitable for crystallization trials to generate crystals that will enable adequate diffraction have now been produced [X. C. Yu et al. (1997) Identification of N-linked glycosylation sites in human testis angiotensin-converting enzyme and expression of an active deglycosylated form. J. Biol. Chem. 272: 3511-3519].

Using the three-dimensional crystal structure coordinates of the N and C domains of ACE, domain-selective ACE inhibitors will be designed by structure-guided techniques, including molecular modeling and computational chemistry, and then synthesized. Inhibitor designs will be refined by testing the specificity and potency of compounds by biochemical and cell-based assays using recombinant wild-type and mutant ACE constructs.

Drug design will be further optimized by co-crystallizations of novel, domain-selective inhibitors with ACE proteins, including full-length and isolated N- and C-domain constructs. Promising candidates will be selected for further study, including initial efficacy and safety studies in appropriate animal models. One or more lead candidates will then be selected for more detailed pharmacological evaluation and toxicology testing. The best candidate will be designated for drug development, at which time phase I clinical trials will be commenced. Phase II(a) trials will be performed in hypertensive patients to evaluate safety and efficacy in this population.

In addition to hypertension, novel, domain-selective ACE inhibitors, designed and synthesized as disclosed in this invention, can be developed for the treatment of a variety of human disease states, including but not limited to: congestive heart failure; left ventricular dysfunction; atherosclerosis and complications of atherosclerotic disease; prevention of stroke, myocardial infarction and other vascular ischemic events; type 2 diabetes mellitus, insulin resistance, and the metabolic syndrome; and prevention and treatment of progressive renal impairment and end-stage renal disease [M. E. Khalil et al. (2001) A remarkable medical story: benefits of angiotensin-converting enzyme inhibitors in cardiac patients. J. Am. Coll. Cardiol. 37: 1757-1764; V. Dzau et al. (2001) The relevance of tissue angiotensin-converting enzyme: manifestations in mechanistic and endpoint data. Am. J. Cardiol. 88 (9 Suppl.): 1L-20L; N. Watson & M. Sandler (1991) Effects of captorpil on glucose tolerance in elderly patients with congestive heart failure. Curr. Med. Res. Opin. 12: 374-378]. Further, N-domain-selective ACE inhibitors are expected to manifest novel therapeutic activities, such as inhibition of hematopoiesis, useful in the treatment of certain blood disorders [R. Plata et al. (2002) Angiotensin-converting-enzyme inhibition therapy in altitude polycythaemia: a prospective randomised trial. Lancet 359: 663-666].

It can be seen that there is a real and continuing need for next-generation ACE inhibitors that are highly domain-selective. These inhibitors will improve the therapeutic efficacy of ACE inhibition and improve the side effect profile.

Such next-generation, domain-selective ACE inhibitors can, as disclosed in this invention, be designed and synthesized by use of the three-dimensional crystal structure coordinates of ACE, using the techniques of structure-guided drug design. This invention has as its primary objective the fulfillment of this need.

Another object of the present invention is to use the methods of molecular modeling, computational chemistry, chemigenomics, and structure-guided drug design to design and synthesize N- and C-domain-selective ACE inhibitors, based on the precise structural information provided by the three-dimensional crystal structure coordinates. The structure-guided drug design approach will include, but is not limited to, optimizing inhibitor backbone and side-chain geometries and functionalities such that the drug compound will bind with greater affinity and specificity to the N- and C-domain active sites and their respective binding pockets. Use of this structure-guided drug design approach will enable discrimination between the N- and C-domain active sites by targeted compounds, such that N-domain-selective inhibitors will bind with ≧2 orders of magnitude greater affinity to the N-domain active site versus the C-domain active site, and vice versa.

Another object of the present invention is to use this structure-guided drug design approach to design and incorporate novel and more effective zinc-binding ligands into the N- and C-domain-selective ACE inhibitors.

Another object of the present invention is to use this structure-guided drug design approach to design and synthesize novel and more effective N- and C-domain-selective ACE inhibitors that inhibit the N- and C-domain active sites by an irreversible inhibition mechanism, such as by alkylation or nucleophilic addition.

Another object of the present invention is to use this structure-guided drug design approach to design and synthesize novel and more effective combined (non-selective) ACE inhibitors that inhibit both the N- and C-domain active sites by an irreversible inhibition mechanism, such as by alkylation or nucleophilic addition.

Another object of the present invention is to refine the use of this structure-guided drug design approach to design and synthesize novel and more effective N- and C-domain-selective ACE inhibitors, and novel and more effective combined (non-selective) ACE inhibitors that inhibit both the N- and C-domain active sites, by performing co-crystallizations of novel ACE inhibitors with ACE proteins, either full-length or isolated N and C domains.

Still another object of the present invention is to develop novel and more effective N- and C-domain-selective ACE inhibitors, and novel and more effective combined (non-selective) ACE inhibitors that inhibit both the N- and C-domain active sites, designed and synthesized by the structure-guided drug design approach described here, for the treatment of a variety of human disease states, including but not limited to: hypertension; congestive heart failure; left ventricular dysfunction; atherosclerosis and complications of atherosclerotic disease; prevention of stroke, myocardial infarction and other vascular ischemic events; prevention and treatment of type 2 diabetes, insulin resistance, and metabolic syndrome; prevention and treatment of progressive renal impairment and end-stage renal disease; and polycythemia.

The means and manner of accomplishing each of the above objectives will become apparent from the detailed description of the invention which follows.

### SUMMARY OF THE INVENTION

ACE is an important drug target in cardiovascular and other diseases, and ACE inhibitors are an important class of therapeutic agents. However, in significant numbers of patients, treatment with current-generation ACE inhibitors produces side effects requiring discontinuation of the drug, or results in loss of efficacy over time. Moreover, it is now known that ACE consists of two, independent domains, the N and C domains, each of which contains its own, independent active site with distinct catalytic properties. It has now been discovered that next-generation ACE inhibitors with improved efficacy and side effect profiles can be developed by designing and synthesizing N- and C-domain-selective ACE inhibitors by structure-guided drug design using the three-dimensional crystal structure coordinates of the N and C domains of ACE.

### DETAILED DESCRIPTION OF THE INVENTION

Over the past 20 years, the importance of ACE and the RAS in cardiovascular physiology and disease have become firmly established, mainly as a result of the use of drugs that block various components of this system, including ACE inhibitors, angiotensin II receptor blockers, and aldosterone antagonists. These drugs are now first-line treatments for hypertension (high blood pressure), heart failure, prevention of vascular events (mainly heart attack and stroke), and slowing kidney disease due to hypertension or diabetes. The involvement of ACE and the RAS in so many different cardiovascular diseases is explained by the fact that the peptide hormone angiotensin II has numerous biological effects [M. Ruiz-Ortega et al. (2001) Role of the renin-angiotensin system in vascular diseases: expanding the field. Hypertension 38: 1382-1387].

Angiotensin II is produced by the action of ACE on a precursor called angiotensin I. Angiotensin II is a powerful vasoconstrictor, which means it causes blood vessels to narrow, which raises blood pressure. Angiotensin II also stimulates the release of the hormone aldosterone from the adrenal glands, which in turn signals the kidneys to retain salt and water, which further raises blood pressure. Furthermore, angiotensin II acts as a growth factor that stimulates thickening of the blood vessel walls, aggravating the process of atherosclerosis, or hardening of the arteries. Therefore, drugs acting on ACE and the RAS reduce blood pressure, improve the function of the heart, and slow down the progression of atherosclerosis and kidney disease. However, angiotensin II is not the only peptide metabolized by ACE.

ACE also acts on other peptide hormones, notably bradykinin, which has the opposite effect of angiotensin II; that is, it is a vasodilator. When ACE is inhibited, this results not only in reduced levels of angiotensin II (therefore, less vasoconstriction) but also in increased levels of bradykinin (therefore, more vasodilatation), which means there is an even greater lowering of blood pressure than if the effect was on angiotensin II alone. Moreover, bradykinin also has anti-inflammatory effects, all of which explains why ACE inhibitors have a different therapeutic profile than drugs that only block the angiotensin II receptor [Dzau et al. (2001); Unger (2002)].

Ten years after the first-generation ACE inhibitors were developed, the ACE gene was finally cloned and the sequence of the enzyme determined. Surprisingly, this revealed that the major form of the enzyme, so-called “somatic ACE,” consists of two similar but non-identical halves, referred to as the N and C domains, each of which contains its own active site. This means that contrary to everything that was believed about ACE before (including the assumptions used in the design of the first-generation ACE inhibitors), ACE is a “double-barreled” enzyme with likely multiple functions that extend beyond what we currently understand. The main function of the C domain appears to be to act on the peptides already discussed, namely angiotensin II and bradykinin. However, the N domain is clearly different, acting specifically on peptides that have nothing to do with blood pressure, such as luteinizing hormone-releasing hormone (LHRH) and the small peptide N-Ac-SDKP, which is an inhibitor of hematopoiesis (production of red blood cells). [F. Soubrier et al. (1988) Two active centers in human angiotensin I-converting enzyme revealed by molecular cloning. Proc. Natl. Acad. Sci. USA 85: 9386-9390; M. R. W. Ehlers et al. (1989) Molecular cloning of human testicular angiotensin-converting enzyme: the testis isozyme is identical to the C-terminal half of endothelial angiotensin-converting enzyme. Proc. Natl. Acad. Sci. USA 86: 7741-7745; M. R. W. Ehlers & J. F. Riordan (1991) Angiotensin-converting enzyme: zinc- and inhibitor-binding stoichiometries of the somatic and testis isozymes. Biochemistry 30: 7118-7126.]

Therefore, as our knowledge of ACE and the RAS has increased it is becoming clear that this system is not only involved in regulating blood pressure. Drugs that impact ACE and the RAS are useful in controlling blood pressure and in treating a broad spectrum of cardiovascular diseases. Beyond that, however, there are novel therapeutic applications, which could include polycythemia (excess red blood cells). Moreover, current-generation ACE inhibitors produce side effects that are likely related to effects on peptides other than angiotensin II. To address these issues requires the design of more selective inhibitors (i.e., N- and C-domain-selective inhibitors), which in turn requires specific structural information. This invention relates to the use of the three-dimensional crystal structure of the N and C domains of ACE to design such inhibitors.

Work on the current-generation ACE inhibitors first began in 1967 at the Squibb Institute for Medical Research and ended 10 years later in the synthesis of captopril, the first orally active, therapeutically useful ACE inhibitor, which is still in use today. The development of captopril was made possible by two discoveries: (1) venom peptides from the Brazilian pit viper inhibit ACE, and (2) ACE is a zinc-dependent enzyme similar to carboxypeptidase-A (CPDA), one of the few enzymes for which an x-ray structure was available at the time. Therefore, although there was no structural information on ACE itself, the information from the venom peptides and CPDA enabled a limited rational design approach that was ultimately successful [M. A. Ondetti et al. (1977) Design of specific inhibitors of angiotensin-converting enzyme: new class of orally active antihypertensive agents. Science 196: 441-444; Cushman & Ondetti (1999)].

Captopril is very effective, but it has the specific disadvantage of a bad taste and chemical instability, due the presence of a thiol group. Captopril was followed by the development of enalapril and lisinopril by Merck after it was shown that several different functional groups could substitute for the thiol in captopril to bind to the active-site zinc in ACE, a critical feature required for the potency and specificity of ACE inhibitors [B. Holmquist & B. L. Vallee (1979) Metal-coordinating substrate analogs as inhibitors of metalloenzymes. Proc. Natl. Acad. Sci. USA 76: 6216-6220; A. A. Patchett et al. (1980) A new class of angiotensin-converting enzyme inhibitors. Nature 288: 280-283]. The work by Holmquist & Vallee not only foreshadowed the Merck compounds, which use a carboxylate for binding to the zinc, but paved the way for subsequent inhibitors using phosphinic acid and hydroxamate zinc-binding groups. Ultimately, more than ten ACE inhibitors were developed, but all were based on the original concepts that guided the design of captopril.

All current-generation ACE inhibitors are similar in their efficacy and side effect profiles, with minor differences in potency and pharmacokinetic properties. The principal side effects include cough and various skin reactions, of which the most serious is life-threatening angioedema; the overall incidence of side effects is estimated at 28% [Steckelings et al. (2001)]. More recently, ACE inhibitors have also been shown to cause mild to moderate anemia. These side effects are likely due to effects of ACE inhibitors on peptides other than angiotensin II, including peptides such as bradykinin, LHRH, N—Ac-SDKP, and substance P, some of which are preferentially or exclusively hydrolyzed by the N domain of ACE.

We have now developed the concept that next-generation ACE inhibitors are required that selectively and potently inhibit either the N or the C domain. Based on what we know today, inhibitors of the C domain will have effects on cardiovascular function similar to those of current-generation ACE inhibitors, but with improved side effect profiles. Moreover, we cannot rule out the possibility that C-domain-selective inhibitors will show a therapeutic spectrum different from current-generation inhibitors, all of which are essentially mixed N and C domain inhibitors. This, together with reduced side effects will enable clear market differentiation. Pure N-domain-selective inhibitors will likely represent a new therapeutic class addressing new markets, including diseases such as polycythemia.

The rational design of potent and specific domain-selective ACE inhibitors has heretofore not been accomplished. The inventors have discovered that the rational design of potent and specific domain-selective ACE inhibitors can be accomplished by structure-guided drug design using the three-dimensional structural coordinates of the ACE N and C domain crystal structures.

The terms “angiotensin-converting enzyme” and “ACE” as used in the context of the present invention can be comprised of full-length wild-type ACE, either the somatic or the testis isoforms, or of various fragments of ACE proteins, notably the isolated N and C domains of the enzyme or derivatives thereof, or of said angiotensin-converting enzyme proteins that contain one or more site-specific or regional mutations, deletions, truncations, insertions, glycosylation changes, or other modifications that facilitate or enhance protein expression, purification, crystallization, x-ray diffraction, or x-ray structure determination or refinement. It is important to note that angiotensin-converting enzyme or ACE is also referred to in the literature as “angiotensin I-converting enzyme,” “converting enzyme,” “dipeptidyl carboxypeptidase,” “petidyldipeptide hydrolase,” or “kininase II;” these terms are all synonymous with angiotensin-converting enzyme and ACE, and this enzyme is classified by the International Union of Biochemists as EC 3.4.15.1 [M. R. W. Ehlers & J. F. Riordan (1990) Angiotensin-converting enzyme. Biochemistry and molecular biology. In Hypertension: Pathophysiology, Diagnosis, and Management (J. H. Laragh & B. M. Brenner, eds.), pp. 1217-1231, Raven Press, New York]. Two forms or isoforms of ACE are known: somatic ACE (also referred to as endothelial or lung ACE) and testis ACE (also referred to as testicular or germinal ACE). Cloning of the ACE gene cDNA revealed that there is a single ACE gene that generates two distinct mRNAs, the somatic ACE mRNA and the testis ACE mRNA, by the use of tissue-specific promoter sites. Further, it was found that the somatic form of ACE consists of two homologous domains arranged in tandem in a single polypeptide chain, termed the N and C domains (referring to their N- and C-terminal locations, respectively, in the polypeptide), and each domain contains an active site characterized by the classic HEXXH zinc-binding motif of metallopeptidases and the presence of 1 zinc atom per active site. Moreover, the testis form of ACE consists of only a single domain, which is identical to the C domain in somatic ACE. [Soubrier et al. (1988); Ehlers et al. (1989); Ehlers & Riordan (1991).]

The ACE crystal structure has long been a holy grail among both industry and academic researchers interested in ACE as a therapeutic target and as a physiologically important enzyme. Efforts to crystallize ACE and determine its structure have been going on since the late 1980s in numerous laboratories and all have failed. The key problems have been to obtain ACE in a form and in quantities sufficient to facilitate crystallization trials, and then to establish the crystallization conditions that will yield crystals of a quality suitable for x-ray diffraction. These problems can now be solved, enabling the determination of the crystal structure at high resolution [Yu et al. (1997)].

It is not uncommon that large proteins are difficult to crystallize. The common somatic form of ACE contains more than 1,400 amino acids, and numerous complicated sugar residues, which hinder crystallization even further. To overcome these problems, efforts can be focused on the isolated C domain of ACE (or the so-called testis form of ACE, which is identical to the C domain of somatic ACE). All unnecessary sequence can be trimmed away and the enzyme expressed in the presence of special glycosylation inhibitors, which will produce a form of the enzyme that is optimal for protein crystallization. The actual crystallization involves a number of trials using different conditions until suitable crystals are grown.

The ACE crystals are then exposed to high-energy x-rays in a synchrotron source, generating so-called diffraction patterns. These diffraction patterns are produced for untreated crystals and crystals soaked in heavy metals, which is required to solve the crystal structure. Moreover, the ACE proteins are also co-crystallized with current-generation ACE inhibitors, such as captopril, enalapril, and lisinopril. The diffraction data are then processed by sophisticated computer programs, which produce a precise three-dimensional picture of the protein, the so-called crystal structure. This structure reveals the exact shape of ACE, how it is regulated, and how the active site acts on the peptide substrates that are its target—this last part is facilitated by the co-crystallization with inhibitors. The precise geometries of the active site sequence, HEMGH, the catalytic zinc atom, the 3^{rd} zinc ligand, and the residues providing additional substrate binding pockets will be the key to future structure-guided drug design, particularly the specific differences between the N- and C-domain active sites.

With the crystal structure of the C domain in hand, the structure of the N domain can be solved more rapidly. The N domain is known to be homologous to the C domain, both in terms of the sequence of amino acids and the presence of sugars, and therefore a similar crystallization strategy can be used. Moreover, once the crystals are available, the x-ray diffraction data are much more quickly and easily analyzed because the C domain structure will serve as a template.

Structure-guided drug design is the process whereby drugs are rationally designed or “built” to fit precisely into a biological target, usually an enzyme or a receptor, which is then activated or inhibited by the drug. This approach is different from the conventional approach of serendipity or random high-throughput screening of compound libraries. However, structure-based drug design requires detailed knowledge of the shape of the drug target, especially the active site or the receptor binding pocket. If the three-dimensional structure of the protein is known, this information can be used directly for the design of new drugs.

With the use of the three-dimensional structures of the ACE N and C domains, novel, domain-selective inhibitors of ACE can designed and tested by the use of computer modeling, using specialized docking programs (e.g., GRAM, DOCK, or AUTODOCK). This procedure includes computer fitting of potential ACE inhibitors to determine how well the shape and the chemical structure of the potential inhibitor will bind to the active site of the enzyme (“Virtual Ligand Screening”). Computer programs can also be used to estimate the attraction, repulsion, and steric hindrance of the N- and C-domains with different inhibitors.

Initially, compounds known to bind ACE, for example lisinopril, which binds to the HEMGH zinc binding motif, can be systematically modified by computer modeling programs until one or more promising potential analogues are identified. Such analysis has been shown to be effective in the development of HIV protease inhibitors. Alternatively, a potential inhibitor will be obtained by first screening commercially available libraries of small molecules, or by screening a random peptide library produced by recombinant bacteriophage. A molecule selected in this manner can then be systematically modified by the computer modeling programs.

Depending on the route by which potential inhibitors are identified, the compound can either be available from commercial libraries of compounds, or the potential inhibitor can be synthesized de novo. De novo synthesis of one or more specific compounds is reasonable in the art of drug design. The potential inhibitors can then be tested in standard binding assays with ACE or an active fragment of ACE, either the N or the C domain, generated by recombinant DNA technology.

The information generated by structure-guided drug design using computer modeling and computational chemistry can be used to synthesize novel domain-selective ACE inhibitors. The objective is to create new inhibitors that are highly selective for either the N or the C domain (i.e., the difference in binding to one domain vs. the other domain should be at least 100-1000 fold). Moreover, new classes of ACE inhibitors can be created by introducing novel zinc-binding groups. The majority of current-generation ACE inhibitors use a carboxyl function to bind the zinc, and a few use thiol or phosphinic acid groups.

Alternative zinc-binding groups could be hydroxamic or boronic acids, which have been shown to be effective in matrix metalloproteinases and dipeptidyl carboxypeptidase, respectively, but have not yet been shown to be effective in ACE inhibitors. Other zinc-binding groups include phosphonates, phosphoramides, guanidinium, sulfates, vanidates, and silanols and silanediols [M. wa Mutahi et al. (2002) Silicon-based metalloprotease inhibitors: synthesis and evaluation of silanol and silanediol peptide analogues as inhibitors of angiotensin-converting enzyme. J. Am. Chem. Soc. 124: 7363-7375]. Moreover, structure-guided drug design will enable the feasibility of designing irreversible inhibitors to be examined, which has not been reported previously. By a combination of these approaches the present invention discloses that compounds can be designed and synthesized that (1) are highly selective for the N- or C-domain active sites; (2) have novel pharmacological spectra because of domain selectivity; and (3) have improved side effect profiles.

One of the key advantages of this structure-guided design approach for the synthesis of 2^{nd}-generation, domain-selective ACE inhibitors is that current-generation inhibitors can be used as a template or backbone on which to build new compounds with altered, domain-selective side chains and functionalities. This substantially reduces the lead time and risk associated with the rational design approach, both in terms of producing real compounds that will work and in terms of the extensive pharmacology-toxicology knowledge base that has accumulated for current-generation inhibitors. For example, it is already known that some ACE inhibitors display different inhibitory potencies toward the two active sites. Captopril is 15 times more potent at inhibiting the C-domain than the N-domain, whereas the phosphinic peptide Ac-Asp-Phe-Ψ(PO₂—CH₂)-Ala-Ala is a far better inhibitor of the N-domain. Using the three-dimensional crystal structure coordinates of the ACE N and C domains, this invention enables those skilled in the art to rapidly build on these concepts to develop highly selective compounds by rational design rather than the random empirical approach used to date.

The synthesis of modified inhibitors (i.e., inhibitors based on known compounds) can be carried out using previously reported methods, as far as possible. These can be used for co-crystallization experiments and structure determination to assess the contacts between the active site of the enzyme and different inhibitors, and can thus provide additional information that can ultimately aid in the design process of new domain-selective ACE inhibitors. Novel inhibitors can be synthesized on the basis of the modeling and crystallographic data. A parallel combinatorial approach can be used to synthesize these new compounds, either as a mixture of compounds or as a library of individual compounds.

Following the initial structure-guided design procedure, a process of lead optimization can be undertaken, which can consist of two phases:

Inhibitor design refinement. A key advantage disclosed in this invention in optimizing lead compounds that are domain-selective ACE inhibitors is to co-crystallize novel compounds with the ACE protein, either the C or the N domain. Co-crystallization is crucial to the process of continuous refinement of inhibitor binding potency and domain selectivity, because it provides “real-time” feedback to the theoretical designs generated during molecular modeling. Thus there is an iterative process whereby designs generated by molecular modeling are synthesized, and the actual compounds can then be co-crystallized with the ACE N or C domain to generate new ACE-inhibitor crystal structures, which in turn can be fed back to optimize the molecular modeling.

Compounds that emerge from this iterative co-crystallization screening process can be further evaluated for their potency and domain-selectivity by measuring how tightly and specifically they bind to either the C or the N domain. These experiments are done in vitro with isolated, recombinant C or N domain, and with ACE constructs expressed in genetically engineered cells. The most promising compounds can then, in turn, be evaluated for their ability to inhibit ACE in vivo in suitable animal models.

Preliminary assessment of drug-like characteristics. Prior to formal preclinical pharmacology and toxicology testing, promising lead candidates can be pre-screened for “drug-like” characteristics by both virtual and experimental methods that can predict the drug's ADMET properties, i.e., its Absorption, Distribution, Metabolism, Elimination, and Toxicology properties. Although these predictive methods are not 100% accurate, they can help narrow down the number of candidates that are designated for further drug development. Predictive modeling of ADMET properties can be performed in parallel with the drug design process and is a means for optimizing the drug-like qualities of compounds. Ideally, drugs should have good oral absorption, low first-pass metabolism in the liver, long half life, no toxic secondary metabolites, predictable hepatic or renal elimination, and no obvious chemistry-related toxicity.

Drug development. The most promising C-domain-selective and N-domain-selective inhibitors generated by the drug design and ADMET screening process can be advanced into formal drug development. This can entail preclinical pharmacology and toxicology testing. The pharmacology testing can include efficacy testing (pharmacodynamics) in an animal model of hypertension, basic pharmacokinetics (i.e., ADME parameters), and acute cardiovascular safety pharmacology. Toxicology testing can entail repeated-dose toxicity testing at up to 50-fold the intended human dose for up to 9 months in two species, usually rat and dog.

Following preclinical evaluation, a phase I clinical study can be conducted in normal human volunteers, for the purpose of evaluating safety and tolerability and basic PK parameters. After satisfactory completion of phase I studies, the lead C-domain-selective and N-domain-selective inhibitors can be evaluated for preliminary efficacy (proof of concept) in phase II studies in hypertensive patients. Additional phase II and phase III studies can be conducted in patients with congestive heart failure or renal insufficiency (proteinuria), or in novel indications as determined by preclinical testing.

### SPECIFIC EXAMPLES

### Example 1

Production of Recombinant ACE Proteins Suitable for Crystallization

Construction of Expression Vectors. pEE-ACEΔ36NJ encodes human tACE that lacks the heavily O-glycosylated, 36-residue N-terminal sequence and is truncated after Ser⁶²⁵, thereby lacking most of the juxtamembrane stalk and the transmembrane and cytoplasmic domains, and is constructed as follows [Yu et al.(1997)]. The 5′-half of the ACE cDNA in the plasmid pLEN-ACE-JMΔ24 is excised by digestion with BamHI and NheI and replaced with the similarly digested fragment from plasmid pLEN-ACEΔ36N. pLEN-ACE-JMΔ24 has an engineered EcoRI site at nucleotide (nt) 1984 in the ACE cDNA [M. R. W. Ehlers et al. (1996) Proteolytic release of membrane-bound angiotensin-converting enzyme: role of the juxtamembrane stalk sequence. Biochemistry 35: 9549-9559]. The sequence between nt 1854 (the start of the unique BclI site) and nt 1990 (the end of the codon for Ser⁶²⁵) in the native ACE cDNA is amplified by the polymerase chain reaction, using a 3′-primer that contained two stop codons (TAA and TAG) after the Ser⁶²⁵ codon, followed by an EcoRI site. The recombinant sequence is inserted into the pLEN-ACE_{—}36N/JMΔ24 hybrid cut with BclI and EcoRI, to generate pLEN-ACEΔ36NJ. The ACEΔ36NJ coding sequence is excised by digestion of unique XbaI (generated after first subcloning in pBluescript) and EcoRI sites and inserted into the polylinker of the expression vector pEE14, to generate pEE-ACEΔ36NJ.

Cell Culture and Transfections. CHO-K1 cells stably transfected with pLEN-ACE glycosylation mutants can be grown and maintained in standard media (50% Ham's F-12/50% DME medium supplemented with 20 mM Hepes, pH 7.3) containing 2% fetal bovine serum (heated to 65° C. for 15 min before use) and 40 μM ZnCl₂. In addition, native CHO-K1 cells can be cotransfected with pEE-ACEΔ36NJ (10 μg) and pSV2NEO (1 μg) by the calcium phosphate precipitate method and clones stably resistant to G418 (Geneticin, Gibco-BRL) can be selected and assayed for ACE activity, by published procedures [Ehlers et al.(1996)]. Clones stably expressing pEE-ACEΔ36NJ can be further selected for resistance to methionine sulfoximine and then amplified, as described (S. J. Davis et al. (1995) J. Biol. Chem. 270, 369-375). Methionine sulfoximine-amplified cells can be grown first in GMEM-10 (Gibco-BRL) containing 10% dialyzed fetal bovine serum (FBS) (Gibco-BRL) and 1.5 mM NB-DNJ for 3 days and then re-fed with GMEM-10, 5% dialyzed FBS, 2 mM NB-DNJ. This medium is changed twice over a period of 9 days before harvesting.

Enzyme Purification. Soluble, recombinant tACE (wild-type, ACEΔ36NJ and ACE glycosylation mutants), can be purified from conditioned media by affinity chromatography on a Sepharose-28-lisinopril affinity resin. The protein can be quantitated by amino acid analysis and assayed for activity using the substrate hippuryl-L-histidyl-L-leucine, as described (M. R. W. Ehlers et al. (1991) Proc. Natl. Acad. Sci. USA 88, 1009-1013).

### Example 2

ACE Protein Crystallization

Crystallization. The purified tACE-ACEΔ36J and ACE glycosylation mutants can be stored at −20° C. in 5 mM Hepes, pH 7.3 and 0.1% PMSF. Extensive crystallization trials using commercially available crystal screen conditions (Hampton Research) can be tried. In addition, ammonium sulfate, PEG and MPD matrices can also be tried. Crystal growth can be tried at 16° C. by the vapor diffusion hanging drop method by mixing the protein solution at ˜11.5 mg/ml in 20 mM Hepes, pH 7.3 and 0.1% PMSF with an equal volume of a reservoir solution containing 15% PEG 4000 (Fluka), 50 mM Sodium acetate trihydrate (Sigma Chemical Company) pH 4.7 and 10 μM ZnSO₄.7H₂O (Aldrich Chemical Company). Crystals usually appear within 2 weeks and grow to their maximum size after about one month.

ACE-Inhibitor Co-Crystallization. The tACE-lisinopril (lisinopril dihydrate, Zeneca Pharmaceuticals), -captopril (Fluka), and -enalapril (enalapril maleate, Sigma Chemical Company) inhibitor complexes can be obtained by growing the crystals in the presence of inhibitor. In these experiments the protein solution can be mixed with 10 mM of the inhibitor and mixed with an equal volume of the reservoir solution before setting up the crystallization.

### Example 3

X-ray Diffraction and Structure Determination

X-ray Diffraction Data Collection. Before data collection, all crystals should be flash-cooled at 100 K in a cryoprotectant containing 15% PEG 4000, 50 mM sodium acetate trihydrate at pH 4.7, 10 μM ZnSO₄.7H₂O and 25% glycerol with and without respective inhibitors. All the X-ray data are collected at 100 K at a synchrotron radiation source. Multi-wavelength anomalous diffraction (MAD) data can be collected. Heavy atoms can be identified that are useful in phasing, prepared by soaking the tACE-inhibitor complex crystals for ˜10 to 60 minutes in the presence of 1-5 mM of heavy atom solutions. Raw data images can be indexed and scaled using the DENZO and SCALEPACK modules of the HKL suite [M. Otwinowski & W. Minor (1997). Processing of X-ray diffraction data collected in oscillation mode. Methods Enzymol 276, 307-326].

Structure Determination and Refinement. The crystal structure of tACE-lisinopril complex can be determined by a combination of MAD and MIRAS (Multiple Isomorphous Replacement with Anomalous Scattering) procedures. The position of the catalytic zinc atom can be unambiguously identified using the anomalous difference Patterson maps calculated using diffraction data at peak wavelength. The identified Zn site can be used to obtain the starting phases in each derivative. Double difference Fourier maps calculated using FFT routine in CCP4 program [Collaborative computational project Number 4. The CCP4 Suite: Programs for Protein Crystallography (1994) Acta Crystallogr. D 50, 760-763] may give the first major binding site, and the phases from the combined Zn and first major site can be used to get additional major/minor sites for each derivative. All heavy atom binding sites and the Zn site can be refined to higher resolution by using the program MLPHARE [Collaborative computational project Number 4. The CCP4 Suite: Programs for Protein Crystallography (1994) Acta Crystallogr. D 50, 760-763] and SHARP [E. De La Fortelle & G. Bricogne Maximum-likelihood heavy-atom parameters refinement in the MIR and MAD methods (1997) Methods Enzymol. 276, 472-494]. The overall figure of merit from SHARP can be improved by iterative solvent flattening, phase combination and phase extension with the program SOLOMON [J. P. Abrahams & A. G. W. Leslie (1996) Methods used in structure determination of bovine mitochondrial F1 ATPase. Acta Crystallogr. D 52, 110-119]. Model building can be carried out manually using the program O [T. A. Jones et al. (1991) Improved methods for building protein models in electron density maps and the location of errors in these models. Acta Crystallogr. A 47, 110-119]. Refinement of the model can be carried out using the program CNS [A. T. Brünger et al. (1998) Crystallography & NMR System: A new software suite for macromolecular structure determination. Acta Crystallogr. D 54, 905-921]. During the final stages of refinement water molecules, the zinc ion and the inhibitor molecule can be inserted in the respective structure.

### Example 4

Structure-guided Design of Domain-selective ACE Inhibitors Molecular Modeling. A prerequisite for structure-based drug design is an understanding of the principles of molecular recognition in protein-ligand (molecule that binds to the protein) complexes. If the three-dimensional structure of the protein is known, this information can be directly exploited for the retrieval and design of new ligands. The determination of the three-dimensional structure of the ACE N and C domains will permit potential domain-selective inhibitors of ACE to be examined by the use of computer modeling using docking programs such as GRAM, DOCK, or AUTODOCK. This procedure can include computational fitting of potential domain-selective ACE inhibitors to determine how well the shape and the chemical structure of the potential inhibitor will bind to the active site of the N or the C domain of ACE. Computer programs can also be used to estimate the attraction, repulsion, and steric hindrance of the N and C domains with different inhibitors.

Initially, compounds known to bind ACE, for example lisinopril which binds to the HEMGH zinc-binding motif, can be systematically modified by computer modeling programs until one or more promising potential analogues are identified. Such analyses have been shown to be effective in the development of HIV protease inhibitors [Lam et al., Science 263:380-384 (1994); Appelt, Perspectives in Drug Discovery and Design 1:23-48 (1993)]. Alternatively, a potential inhibitor can be obtained by initially screening commercially available libraries of small molecules, or a random peptide library produced by recombinant bacteriophage [Scott and Smith, Science 249:386-390 (1990)]. A molecule selected in this manner can then be systematically modified by computer modeling programs, as described above.

Once a potential inhibitor has been identified it will be selected either from a library of chemicals that are commercially available from most large chemical companies, or alternatively the potential inhibitor can be synthesized de novo. The de novo synthesis of one or even a relatively small group of specific compounds is reasonable in the art of drug design. The potential inhibitor can be placed into a standard binding assay with ACE or an active fragment of ACE, either the N or the C domain, generated by recombinant DNA technology, synthesized by standard peptide synthesis, or classical proteolysis. Alternatively the corresponding full-length proteins, purified from natural sources, such as mammalian, including human, lung, kidney, or testis tissue, or generated recombinantly, such as in CHO cells or COS cells, may be used in these assays.

ACE Domain-directed Computational Chemistry. Differences in catalytic specificity and efficiency between the N- and C-domain active sites are known in the art, but the structural basis for these differences is unknown. With the availability of the three-dimensional crystal structure of the N and C domains of ACE, the structural basis for differences between the two active sites can be understood and domain-selective inhibitors can be rationally designed and synthesized, as provided for in this invention. For example, it is known that the N domain active site is fully activated at chloride concentrations of about 30 mM, whereas the C-domain active site requires about 300 mM, depending on the substrate. Moreover, whereas the C-domain active site generally only cleaves oligopeptides with unblocked C termini at the penultimate C-terminal peptide bond (dipeptidyl carboxypeptidase cleavage), the N-domain active site has been shown to cleave near the N terminus of N- and C-blocked oligopeptides, such as LHRH and AcSDKP.

Therefore, it can be envisaged that there are specific and important differences in the binding pockets and geometries in the C- and N-domain active sites, as well as differences in the chloride binding sites and their effects on the conformation of the domain. These differences can be exploited to guide domain-selective inhibitor design. It may be found, for example, that the S₁′ binding pocket in the C-domain active site [for relevant nomenclature see M. A. Ondetti & D. W. Cushman (1982) Ann. Rev. Biochem. 51: 283-308], is very deep and accommodates a much larger side-chain than the amino-butyl side chain in lisinopril. This can be exploited by introducing a longer or bulkier side chain onto, for example, a lisinopril template molecule, and this can be expected to bind tightly to the C-domain active site but poorly to the N-domain active site. Similarly, important differences may also be found in the S₁ and S₂′ binding pockets between the N- and C-domain active sites, which can be further exploited by structure-guided drug design to develop domain-selective inhibitors. Further, it may be found that the Zn²⁺ ion geometry differs between the two active sites, allowing for the use of domain-selective zinc-ligating functionalities in the inhibitor design.

It may further be found that the COOH-binding active site residue differs between the two active and/or that it may be amenable to the incorporation of a functionality that can covalently modify this residue to produce an irreversible inhibitor design. It has long been assumed that the COOH-binding residue is a positively charged arginine [M. A. Ondetti & D. W. Cushman (1981) in Biochemical Regulation of Blood Pressure (R. L. Soffer, ed.), pp. 165-204, Wiley, N.Y.], but it may also be a lysine in either or both of the active sites. If it is a lysine, this would present a clear opportunity for covalent modification, by, for example, the introduction of an alkyl halide or halo-ketone functionality into the inhibitors that can alkylate the lysine amine, or α-ketone or aldehyde that can form a Schiff's base with the lysine amine, or the use of activated ester or thioester groups, or other modified carboxyl groups susceptible to nucleophilic attack.

### Example 5

Synthesis of Domain-selective ACE Inhibitors

Some current-generation ACE inhibitors display different inhibitory potencies toward the two active sites. Captopril is 15 times more potent at inhibiting the C domain than the N domain, whereas a phosphinic peptide, Ac-Asp-Phe-Ψ(PO₂—CH₂)-Ala-Ala-NH₂, is a far better inhibitor of the N domain [C. Junot et al. (2001) RXP 407, a selective inhibitor of the N-domain of angiotensin I-converting enzyme, blocks in vivo the degradation of hemoregulatory peptide acetyl-Ser-Asp-Lys-Pro with no effect on angiotensin I hydrolysis, J. Pharmacol. Exp. Therapeut. 297: 606-611]. The requirements for inhibition of the N domain may therefore be the presence of a C-terminal amide group, Asp in the P₂ position, and an N-acetyl group at the N terminus. Compounds with these structural features tend to bind repulsively at the C domain but are well tolerated by the N domain. Fosinopril, also a phosphinic inhibitor, is more N-selective and it might be that the phosphinic moiety plays a role in N domain specificity for this type of compound. Angiotensin (1-7) [Asp¹-Pro⁷] and the blocked synthetic peptide Bz-Phe Ψ (CO—CH₂)Gly-Pro (keto-ACE) were shown to be more effective inhibitors of the C domain [R. G. Almquist et al. (1980) Synthesis and biological activity of ketomethylene analogue of a tripeptide inhibitor of angiotensin converting enzyme. J. Med. Chem. 23: 1392-1398; P. A. Deddish et al. (1998) N-domain-specific substrate and C-domain inhibitors of angiotensin-converting enzyme: angiotensin (1-7) and keto-ACE. Hypertension 31: 912-917]. Although current-generation ACE inhibitors bind to both domains, they may differ in their affinities, depending on their structure, primarily because of differences in dissociation rates from the two active sites. Most of these partial domain-selective inhibitors were stumbled upon by screening the N- and C-domain active sites with a variety of inhibitors by random screening, whereas our invention will exploit differences in the three-dimensional crystal structures of the two active sites.

The synthesis of representative known and modified inhibitors can be carried out using previously reported methodology, as far as possible. These can be used for co-crystallization experiments and structure determinations to assess the contacts between the active site of the N and C domains and different inhibitors, and thus provide additional information that can ultimately aid in the design process of new domain-selective ACE inhibitors. Novel inhibitors will be synthesized on the basis of the modeling and crystallographic data. A parallel combinatorial approach can be used to synthesize these new compounds, either as a mixture of compounds or as a library of individual compounds.

For the first phase, inhibitors of ACE that are not commercially available can be synthesized for co-crystallization studies. Below are the compounds envisaged for this purpose. As mentioned above, keto-ACE is a C-domain specific inhibitor and thus would give insight into binding at this particular active site of ACE.

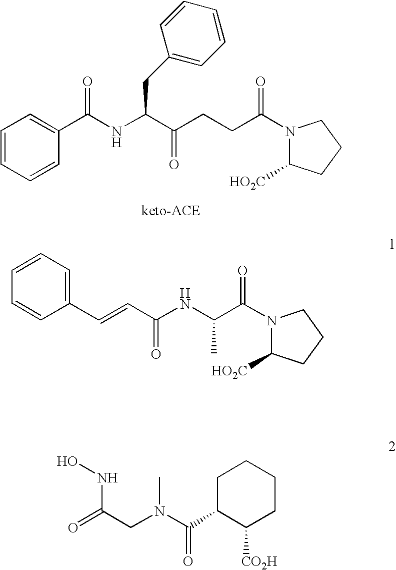

Compound 1 is an a,b-unsaturated amide that is envisaged to react with a catalytic nucleophile at the active site of the enzyme to give a Michael-type addition product, thus modifying the nucleophile covalently. This compound inhibits Hip-His-Leu hydrolysis by rabbit liver ACE in vitro with an IC₅₀ of 0.23 mM [H.-Y. P. Choo et al. (2000) Design and synthesis of α,β-unsaturated carbonyl compounds as potential ACE inhibitors. Eur. J. Med. Chem. 35: 643-648]. Mechanistically, coordination of the Zn²⁺ to the carbonyl next to the olefinic bond with a carboxylate from Glu127 acting as the nucleophile will lead to irreversible inhibition of the enzyme. The irreversible nature of the inhibition was experimentally supported by the time-dependent loss of enzymatic activity, where ˜50% of enzyme activity remained after 10 minutes of incubation with 0.4 mM of inhibitor 1. Enzyme activity could not be recovered after dialysis, implying covalent modification of the enzyme. An advantage of such enzyme inactivators is their potential long duration of action.

Three molecular characteristics, a Zn-ligand group (hydroxamate), a shifted N-alkylated amide function, and a 1,2-cyclohexanedicarboxylic acid moiety were combined to give the non-amino acid structure (2), which met ACE active site binding requirements as effectively as the amino acid structures of classical ACE inhibitors. Hydroxamic derivative (2) is a competitive inhibitor (Kᵢ=2.7±0.2 nM) of ACE with an IC₅₀ of 7.0 nM with Hip-Gly-Gly as substrate, while that for captopril using the same assay was 3.0 nM. R-configuration at cyclohexane C-2 was a stereochemical feature required for activity. The hydroxamic acid moiety played a dominant role in the affinity of this type of compounds, as the benzylhydroxamate precursors were inactive against ACE in vitro [L. Turbanti et al. (1993) 1,2-Cyclomethylenecarboxylic monoamide hydroxamic derivatives. A novel class of non-amino acid angiotensin converting enzyme inhibitors. J. Med. Chem. 36: 699-707].

It is noteworthy that the classical (current-generation) ACE inhibitors contain a 3-phenyl ethylene moiety at the N terminus and this is missing from the compounds described above. Thus, it is envisaged that incorporating this feature into compound 2 will lead to a novel structures, which could provide stronger inhibition of the enzyme. Another novel structure can be synthesized by transforming the phenylalanine in keto-ACE to an a,b-unsaturated amide moiety, a derivative that may covalently modify the C-domain active site of ACE.

After chemical synthesis of the aforementioned inhibitors, keto-ACE and compounds 1 and 2 can be co-crystallized with ACE. The compounds can be evaluated for their inhibitory potency against the ACE N and C domains before crystallization studies are undertaken. The characteristics of these inhibitors towards the two ACE domains can be investigated separately by selecting appropriate substrates. Inhibition studies with these substrates on testis ACE, which only contains the C domain, or on ACE from deletion mutants for either the N or C domain, may be used to determine to what extent they interact with either domain.

It can be seen from the above examples which are illustrative only of aspects of the present invention that it accomplishes all of its stated objectives. Importantly, these examples should in no way be taken as a limitation of the teachings or the disclosure or the range or equivalence of the present invention, as they are exemplary only.

We claim:

1. A method for the design and synthesis of domain-selective inhibitors of angiotensin-converting enzyme (ACE) that are highly selective for either the N domain or the C domain of ACE, by the use of the three-dimensional crystal structure of an angiotensin-converting enzyme, comprising the steps of:
a. using a three-dimensional structure of said enzyme as defined by atomic coordinates of an angiotensin-converting enzyme;
b. employing said three-dimensional structure to design or select said inhibitors;
c. synthesizing said inhibitors;
d. contacting said N- and C-domain-selective inhibitors with said enzyme in the presence of a substrate to determine the ability of said inhibitors to selectively inhibit the N and C domains, respectively, of ACE; and
e. co-crystallizing said N- and C-domain-selective inhibitors with said enzyme to determine and optimize the ability of said inhibitors to selectively inhibit the N and C domains, respectively, of ACE.

2. A method of claim 1 wherein the three-dimensional crystal structure of ACE is of the full-length, wild-type somatic form of the enzyme, generated recombinantly in CHO cells, COS cells, or other suitable mammalian cells, or other eukaryotic, e.g., yeast, or prokaryotic, e.g., Escherichia coli, cells, or purified from natural sources, such as lung or kidney tissue.

3. A method of claim 1 wherein the three-dimensional crystal structure of ACE is of the full-length, wild-type testis form of the enzyme, generated recombinantly in CHO cells, COS cells, or other suitable mammalian cells, or other eukaryotic, e.g., yeast, or prokaryotic, e.g., Escherichia coli, cells, or purified from natural sources, such as testis tissue.

4. A method of claim 1 wherein the three-dimensional crystal structure of ACE is of the isolated N domain of the somatic form of the enzyme, generated recombinantly in CHO cells, COS cells, or other suitable mammalian cells, or other eukaryotic, e.g., yeast, or prokaryotic, e.g., Escherichia coli, cells, or generated by limited proteolysis of somatic ACE purified from natural sources, such as lung or kidney tissue, or generated by peptide synthesis.

5. A method of claim 1 wherein the three-dimensional crystal structure of ACE is of the isolated C domain of the somatic or testis forms of the enzyme, generated recombinantly in CHO cells, COS cells, or other suitable mammalian cells, or other eukaryotic, e.g., yeast, or prokaryotic, e.g., Escherichia coli, cells, or generated by limited proteolysis of somatic or testis ACE purified from natural sources, such as lung, kidney, or testis tissue, or generated by peptide synthesis.

6. A method of claim 1 wherein said angiotensin-converting enzymes contain one or more site-specific or regional mutations, deletions, truncations, insertions, glycosylation changes, or other modifications that facilitate or enhance protein expression, purification, crystallization, x-ray diffraction, or x-ray structure determination or refinement.

7. A method of claim 1 wherein said N- and C-domain-selective inhibitors are designed and synthesized de novo.

8. A method of claim 1 wherein said N- and C-domain-selective inhibitors are designed and synthesized from one or more known inhibitors.

9. A method of claim 1 wherein said N- or C-domain-selective inhibitors are competitive inhibitors of angiotensin-converting enzyme.

10. A method of claim 1 wherein said N- or C-domain-selective inhibitors are non-competitive, uncompetitive, or irreversible inhibitors of angiotensin-converting enzyme.

11. A method for the design and synthesis of inhibitors of angiotensin-converting enzyme (ACE) that are non-selective and active against both the N domain and the C domain of ACE, by the use of the three-dimensional crystal structure of an angiotensin-converting enzyme, comprising the steps of:
a. using a three-dimensional structure of said enzyme as defined by atomic coordinates of an angiotensin-converting enzyme;
b. employing said three-dimensional structure to design or select said inhibitors;
c. synthesizing said inhibitors;
d. contacting said non-selective inhibitors with said enzyme in the presence of a substrate to determine the ability of said inhibitors to inhibit both the N and C domains, respectively, of ACE; and
e. co-crystallizing said non-selective inhibitors with said enzyme to determine and optimize the ability of said inhibitors to inhibit both the N and C domains, respectively, of ACE.

12. A method of claim 11 wherein the three-dimensional crystal structure of ACE is of the full-length, wild-type somatic form of the enzyme, generated recombinantly in CHO cells, COS cells, or other suitable mammalian cells, or other eukaryotic, e.g., yeast, or prokaryotic, e.g., Escherichia coli, cells, or purified from natural sources, such as lung or kidney tissue.

13. A method of claim 11 wherein the three-dimensional crystal structure of ACE is of the full-length, wild-type testis form of the enzyme, generated recombinantly in CHO cells, COS cells, or other suitable mammalian cells, or other eukatyotic, e.g., yeast, or prokaryotic, e.g., Escherichia coli, cells, or purified from natural sources, such as testis tissue.

14. A method of claim 11 wherein the three-dimensional crystal structure of ACE is of the isolated N domain of the somatic form of the enzyme, generated recombinantly in CHO cells, COS cells, or other suitable mammalian cells, or other eukaryotic, e.g., yeast, or prokaryotic, e.g., Escherichia coli, cells, or generated by limited proteolysis of somatic ACE purified from natural sources, such as lung or kidney tissue, or generated by peptide synthesis.

15. A method of claim 11 wherein the three-dimensional crystal structure of ACE is of the isolated C domain of the somatic or testis forms of the enzyme, generated recombinantly in CHO cells, COS cells, or other suitable mammalian cells, or other eukaryotic, e.g., yeast, or prokaryotic, e.g., Escherichia coli, cells, or generated by limited proteolysis of somatic or testis ACE purified from natural sources, such as lung, kidney, or testis tissue, or generated by peptide synthesis.

16. A method of claim 11 wherein said angiotensin-converting enzymes contain one or more site-specific or regional mutations, deletions, truncations, insertions, glycosylation changes, or other modifications that facilitate or enhance protein expression, purification, crystallization, x-ray diffraction, or x-ray structure determination or refinement.

17. A method of claim 11 wherein said non-selective inhibitors are designed and synthesized de novo.

18. A method of claim 11 wherein said non-selective inhibitors are designed and synthesized from one or more known inhibitors.

19. A method of claim 11 wherein said non-selective inhibitors are competitive inhibitors of angiotensin-converting enzyme.

20. A method of claim 11 wherein said non-selective inhibitors are non-competitive, uncompetitive, or irreversible inhibitors of angiotensin-converting enzyme.
